(19) United States
(12) Patent Application Publication
(10) Pub. No.: US 2026/0310823 A1
(43) Pub. Date: Oct. 8, 2026

(54) APPARATUS FOR AND METHOD OF OBSCURING INFORMATION

(71) Applicant: PROXIMIE LIMITED, Altrincham (GB)

(72) Inventor: Mohamad Mehdi ATIYE, Altrincham (GB)

(21) Appl. No.: 18/998,461

(22) PCT Filed: Jul. 26, 2023
(86) PCT No.: PCT/GB2023/051979

§ 371 Date: Jan. 24, 2025

(30) Foreign Application Priority Data
Jul. 26, 2022 (GB) 2210931.8

(51) Int. Cl. G06F 21/62; G06T 5/50; G06T 5/70; G06T 5/77; G16H 40/67

(52) U.S. Cl. CPC G06F 21/6254; G06T 5/50; G06T 5/70; G06T 5/77; G16H 40/67; G06T 2207/10016; G06T 2207/30004

(57) ABSTRACT

There is provided an apparatus for processing an imaging feed from an imaging device, comprising: a device for transforming the imaging feed; and a communication interface for communicating the imaging feed to a further device; wherein only a transformed imaging feed is transmitted beyond the communication interface.

### TECHNICAL FIELD

The present invention relates to an apparatus for and method of obscuring sensitive information. In particular, the present invention relates to an apparatus for and method of processing an imaging feed from an imaging device to obscure regions as desired.

### BACKGROUND

In a variety of situations, it can be desirable to obscure sensitive information from an imaging feed such as a video stream. For example, in the field of telehealth or telesurgery, patient data, faces or tattoos may be visible. The surgeon or other user may wish to obscure this data for the sake of the privacy of the patient. The present invention is concerned with an improved manner of achieving this.

### SUMMARY OF INVENTION

According to a first aspect of the invention, there is provided apparatus for processing an imaging feed from an imaging device, comprising: a device for transforming the imaging feed; and a communication interface for communicating the imaging feed to a further device; wherein only a transformed imaging feed is transmitted beyond the communication interface.

By only transmitting the transformed imaging feed beyond the communication interface, the security of the apparatus can be improved. The original or ‘untransformed’ imaging feed thus is not distributed and cannot be intercepted. The transformation may be used to obscure regions of the imaging feed, preferably wherein the regions include sensitive information. This means that the regions are not transmitted beyond the communication interface. This can help in ensuring that the transformed imaging feed is immutable (i.e. the original, untransformed imaging feed cannot be recovered). Preferably only an immutably transformed imaging feed is transmitted beyond the communication interface.

Preferably, the further device is external to the communication interface. As used herein the term ‘external’ preferably connotes that the further device is logically external to the communication interface. The communication interface preferably provides an interface between one logical network and external entities. The communication device is preferably internal or local to the imaging device. ‘Local or internal’ preferably connotes that it is provided as part of the same logical network. This may be the case even if they are provided on different hardware, which may be, for example, in an adjacent room. Furthermore, the transformation is preferably performed locally. The apparatus may further comprise a processor for transforming the imaging feed. The processor may be internal to the communication interface in that it is part of the same logical network. The original (untransformed) version of the imaging feed may therefore remain within the same logical network. Preferably, the device for transforming the imaging feed and the communication interface may be provided on the same (self-contained) device. For example, the device for transforming the imaging feed and the communication interface may be provided on the same computer device. In such an instance, the device for transforming the imaging feed may be a processor of the computer device. In such an instance, the communication interface enables the computer device to communicate with further (external) devices. The communication may be via an internet connection (facilitating, for example, communication to the further, external devices in physically remote locations) or via a wired connection (facilitating, for example, communication to further, external devices within a same location, such as a building or room). The logical network may preferably comprise the imaging device. For example, the computer device may be connected to the imaging device or may comprise the imaging device.

The further device may be a server, wherein preferably the server is configured to distribute the transformed imaging feed. As used herein, the term ‘server’ preferably connotes a computer device or computer program configured to manage a centralized resource or service in a network, and/or to manage access to that centralized resource or service. As used herein, the term ‘distribute’ preferably encompasses broadcasting, transmitting, displaying, recording, saving, and/or assembling (although the term is not limited to these actions). For example, the server may receive the transformed imaging feed and then broadcast it to one or more further entities and/or devices. The server may distribute to a network of further entities such as devices.

Preferably, the further device is remote and/or geographically distant from the imaging device and communication interface. As used herein the terms ‘geographically distant’ and ‘remote’ preferably encompass being located in a different country, city, building or even room. The term ‘remote’ preferably connotes being part of a separate logical network. This may encompass being geographically close (for example in the same room) but logically remote (i.e. logically distinct and/or separate).

In preferable implementations, the imaging feed is not recorded prior to transformation by the transforming device. This can improve the security of any information which a user wishes to obscure from the imaging feed, as it cannot be recovered from a stored file.

Preferably, the imaging feed is transmitted from the imaging device to the transforming device without being recorded. As used herein, the term ‘transmitted’ preferably connotes any manner of sending the imaging feed to another entity, including electronic transmission or broadcast. Preferably the imaging feed is transmitted from the imaging device to the transforming device without being recorded by any recording device. More preferably, the imaging feed is transmitted from the imaging device to the transforming device without being broadcast. As such, preferably only a transformed imaging feed may be transmitted or recorded.

The apparatus preferably further comprises means (preferably in the form of an authorization device or module, and/or preferably embodied in a processor and associated memory) for authorizing an entity to control the imaging feed before it is communicated to the further device. This feature may also be provided independently. In such an instance, the entity preferably only receives the transformed imaging feed.

According to a further aspect of the invention, there is provided apparatus for processing an imaging feed from an imaging device, comprising: a communication interface for communicating the imaging feed to a further device; and means (preferably in the form of an authorization device or module, and/or preferably embodied in a processor and associated memory) for authorizing an entity to control the imaging feed before it is communicated to a further device.

Expressed in a different manner, the apparatus may be configured for authorizing an entity to control the imaging feed before it is communicated to a further device.

An entity may be a (human) user or a (computer) device. The ‘authorization device’ may be or be embodied in a module of a computer device or server or a separate and/or distinct device. As used herein, the term ‘control’ preferably encompasses passive actions, such as (but not limited to) accessing and viewing, and active actions such as (but not limited to) editing, manipulating and instructing. The further device may be a server. This can enhance the security of an imaging feed by requiring authorizations to control an imaging feed. This can prevent an external entity from controlling (such as viewing and/or editing and/or manipulating) the imaging feed.

As used herein, the term ‘before’ preferably connotes an earlier position in a communication path and/or at an earlier point in time.

Preferably, the authorizing means is configured for authorizing an entity to view and/or edit the imaging feed before it is communicated to a further device. This may be before a transformation has been applied. This may authorize an entity to view an original or untransformed imaging feed. This may authorize an entity to edit an original or untransformed imaging feed, for example by defining a transformation. These may be separate and/or distinct authorizations.

Preferably, the authorizing means is configured for authorizing an entity to define a transformation applied to the imaging feed before it is communicated to a further device. This may authorize an (internal or external) entity to define a transformation, preferably wherein the transformation is applied before the imaging feed is transmitted beyond the internal device. This may be beyond a communication interface and/or outside of a logical network. The entity authorized to define a transformation may be the transforming device or a further device (for example, a device external to the transforming device).

Preferably, the authorizing means is configured to prohibit an entity being able to control the imaging feed before it is communicated to a further device. This may encompass prohibiting an entity to view and/or edit and/or manipulate the imaging feed. These may be separate and/or distinct authorizations. This may authorize an entity to define and/or edit a transformation applied to the imaging feed.

In preferable implementations, the authorizing means is configured to prohibit an entity being able to view the imaging feed before it is communicated to a further device and to authorize that same entity to define a transformation applied to the imaging feed, preferably before it is communicated to a further device. This may authorize an external entity to define a transformation, while still ensuring that only a transformed imaging feed is transmitted beyond an internal device (for example a logic network, which may preferably contain the imaging device, and may further contain a communication interface for transmitting the feed). For example, an entity may have authorization to define a (new) transformation and/or edit the transformation but still views only a transformed imaging feed. Preferably, only the transformed imaging feed is transmitted (and/or communicated) to the entity, even while the entity is defining a further (new) transformation and/or editing the transformation. Preferably, the entity views a preview of the further (new) transformation and/or edited transformation applied to and/or on the transformed imaging feed (i.e. views the preview of the further (new) transformation and/or edited transformation simultaneously with a current transformation).

In preferable implementations, there may be differential levels of authorization. The authorizing means may define different authorizations and/or authorization levels for different entities. The authorizing means may define different combinations of authorizations for different entities. The different (or differential) levels of authorization may be defined by different combinations of levels of authorizations.

Preferably, the authorizing means is configured to determine a level of authorization of the entity in dependence on its status. As used herein, the term ‘status’ may refer to the relationship of the entity to the imaging feed and/or may refer to an attribute assigned to the entity. For example, a user may be assigned a level of authorization. As another example, a device may be assigned a level of authorization, preferably in dependence of its status as internal or external to the imaging feed (for example internal or external to the logic network of the imaging device and/or communication interface). As another example, an entity may be assigned a level of authorization in dependence of its status as a controller, initiator or participant in a ‘session’. A ‘session’ may refer to an implementation of instructions according to the present invention.

In preferable implementations, the authorizing means is configured to authorize an entity that controls the communication interface to view the imaging feed before it is communicated to a further device. This may authorize the entity to view an original or untransformed imaging feed, yet enable only a transformed imaging feed to be transmitted beyond the communication interface.

In some implementations, the apparatus may further comprise a further communication interface for communicating a further imaging feed to the or a server. This may allow two or more imaging feeds to be communicated to the server. The authorizing means may be configured to define different authorizations for the further communication interface. The authorizing means may be configured to define different authorizations for entities with respect to each imaging feed and/or communication interface.

The transformed imaging feed may comprise the imaging feed with at least one region extracted. The at least one region of the imaging feed may in this manner be completely removed from the imaging feed, such that it cannot be recovered. Such an implementation helps to ensure that the transformation is immutable.

The apparatus may further comprise a processor for: extracting at least one region of the imaging feed and replacing it with at least one region of an alternative image to generate a transformed imaging feed. The processor may thus be configured for transforming the imaging feed. This feature may be provided independently.

According to a further aspect of the invention, there is provided apparatus for processing an imaging feed from an imaging device, comprising: a processor for: extracting at least one region of the imaging feed; and replacing it with at least one region of an alternative image to generate a transformed imaging feed.

This method can advantageously transform the imaging feed in a manner which is immutable. The at least one region of the imaging feed may comprise sensitive information. The at least one region of the imaging feed may in this manner be completely removed from the imaging feed, such that it cannot be recovered. As such, the transformation may be immutable. The processor may thus be configured for transforming the imaging feed.

The ‘alternative image’ may comprise a blank image, an image of uniform pixel value (such as a uniform colour), or an alternative image (for example, displaying a non-uniform display, comprising different pixel values). The alternative image may comprise a processed copy of the imaging feed, for example the alternative image may comprise an obscured copy (for example a pixellated or blurred copy or version) of the imaging feed.

The processing steps may be applied to an image of the imaging feed. The processing steps may further comprise the step of: generating an image of the imaging feed.

The transformed imaging feed may be assembled from a series of transformed images. The processing steps may further comprise performing the steps on an image of the imaging feed. The processing steps may further comprise: generating a transformed imaging feed using the transformed image. The processor may generate the transformed imaging feed using time stamps.

Preferably, the at least one region of the alternative image spatially corresponds to the at least one region of the imaging feed. As used herein, ‘spatially corresponds’ preferably connotes the at least one region of the alternative image has the same configuration (such as size, location and orientation) as the at least one region of the imaging feed. If the alternative image and the imaging feed have the same dimensions, the at least one region of the alternative image may have the same coordinates as the at least one region of the imaging feed.

The alternative image may be an image of the imaging feed, to which the processor is configured to apply a filter. An image of the imaging feed may be considered to be a further imaging feed. The alternative image may thus be a ‘copy’ of the imaging feed. The image may be recorded temporarily. The image may have an image file format, for example a bitmap image file format.

Preferably, the processor is configured to apply the filter to a larger portion of the image than the at least one region of the alternative image. More preferably, the processor is configured to apply the filter in respect of the whole alternative image. This can prevent a reduced quality of the filter at the edges of the at least one region. This can also improve computational efficiency.

Preferably, the filter is an obscuring filter. As used herein, ‘obscuring filter’ preferably connotes any filter which reduces detail of an image. Preferably, the filter is a blurring filter. As used herein, ‘blurring filter’ preferably connotes a filter which blurs the image, thereby reducing clarity and/or detail. Even more preferably, the filter is a gaussian blurring filter. In some implementations, the filter may be a pixelating filter. As used herein, ‘pixelating filter’ preferably connotes a filter which reduces the pixel density, replacing a number of pixels greater than one with a colour value which is an average of the colour values of those pixels. The same apparatus may be configured to applying different filters.

Preferably, the filter may be selected in dependence on a performance metric of the apparatus. In preferable implementations, the performance metric relates to processing capability. As used herein, ‘metric’ preferably connotes a measured and/or calculated numerical or non-numerical value and/or parameter and/or set of values and/or parameters. The performance metric may be determined (for example via measurement or calculation) continuously or intermittently over time. The performance metric may be related to processing speed and/or connection speed. A different filter may be selected over time in dependence on changes to the performance metric. This can help to prevent interruptions to the transformation and/or transmission of the feed.

In preferable implementations, the imaging feed is live, and/or transformation of the imaging feed is performed in real time to generate a live transformed imaging feed. Preferably, the transformation is performed within 500 ms of receiving the imaging feed, preferably within 300 ms, more preferably within 200 ms. As used herein the term ‘live’ preferably encompasses the transmission of the feed beyond the processor (for example, beyond a communication interface) within 500 ms of the imaging feed being generated and/or transmitted to the processor, preferably within 300 ms, more preferably within 200 ms.

The apparatus preferably further comprises a user interface configured to enable a user to define and/or edit a or the transformation applied to the imaging feed. As used herein, a ‘user interface’ preferably connotes any device or apparatus which allows a user to interact with the apparatus of the present invention. Preferably, it allows a user to provide instructions to the processor. The ability to define and/or edit a transformation is preferably in dependence on an authorization and/or status of the entity and/or user.

Preferably, the user interface is configured to enable a user to define the at least one region. The user interface and/or processor is preferably configured to transform the input to and/or interaction with the user interface into computer-implementable instructions, thereby to define the at least one region.

In preferable implementations, the user interface is configured to enable a user to define a location and/or shape and/or size and/or orientation of the at least one region. Preferably, the user interface is configured to enable a user to select a shape. More preferably, the shape can be selected from a list comprising at least a rectangle and an ellipse. These shapes preferably encompass the specific examples of a square and a circle. The shape can be used to define regions to be obscured of the imaging feed. This can enable a user to define these regions quickly and efficiently.

Preferably, the user interface is configured to enable a user to define and/or edit the transformation while the imaging feed is being transmitted. This may enable a user to edit the transformation of an imaging feed while it is being transmitted and/or communicated. This may be subject to authorizations of the user and/or device, as described above. The user interface and/or processor may be configured to translate a user's interactions into computer-implementable instructions. The instructions may define an edited transformation. The instructions may be sent and/or transmitted to the server. The instructions may be sent and/or transmitted to the imaging device and/or associated processor (this may be via the server). Preferably, edits to the transformation are only finalized upon the user inputting a confirmation, preferably into the user interface.

In preferable implementations, the apparatus is further configured to output an alert upon detecting that the imaging device is capable of altering a configuration of the imaging feed. Preferably, this occurs upon detecting that the imaging device is capable of altering pan, tilt and/or zoom settings of the imaging feed. This can alert a user or device that the imaging feed may be reconfigured such that the regions contain different information.

The apparatus preferably further comprises the imaging device. The imaging device may be located in a medical facility. Preferably, the imaging feed is derived from a medical facility. Any feature of the apparatus may be provided in a medical facility. The apparatus may be provided in a medical facility. For example, the medical facility may be a surgical facility. The medical facility may be an operating theatre. The user interface may be provided in the medical facility. The apparatus may comprise more than one imaging feed deriving from different medical facilities. For example, they may derive from different departments internal to a hospital including different operating theatres, laboratories, wards or offices. Alternatively, they may derive from different hospitals, clinics or universities. A user, who may interact with the user interface, may be a medical professional such as a doctor, dentist, nurse, medical student, healthcare assistant, administrator or similar.

According to a further aspect of the invention, there is provided a method of processing an imaging feed from an imaging device, comprising: transforming the imaging feed; and communicating only a transformed imaging feed imaging feed to a further device.

According to a further aspect of the invention, there is provided a method of processing an imaging feed from an imaging device, comprising: communicating the imaging feed to a further device; and authorizing an entity to control the imaging feed before it is communicated to a further device.

According to a further aspect of the invention, there is provided a method of processing an imaging feed from an imaging device, comprising: extracting at least one region of the imaging feed; and replacing it with at least one region of an alternative image to generate a transformed imaging feed.

The methods may further comprise the apparatus features as described. The features of the methods and apparatus as described may be provided in any combination.

As used herein the term ‘apparatus’ preferably encompasses a device or a network of devices. As described herein, each of the ‘devices’ may be provided as a device comprising a processor and associated memory. Alternatively, the devices may be provided as a module of a larger device, wherein the larger device comprises a processor and associated memory in communication with the module.

The invention extends to methods and/or apparatus substantially as herein described with reference to the accompanying drawings.

Any apparatus feature as described herein may also be provided as a method feature, and vice versa. Any feature in one aspect of the invention may be applied to other aspects of the invention, in any appropriate combination. In particular, method aspects may be applied to apparatus aspects, and vice versa. Furthermore, any, some and/or all features in one aspect can be applied to any, some and/or all features in any other aspect, in any appropriate combination.

Furthermore, features implemented in hardware may be implemented in software, and vice versa. Any reference to software and hardware features herein should be construed accordingly.

Any apparatus feature as described herein may also be provided as a method feature, and vice versa. As used herein, means plus function features may be expressed alternatively in terms of their corresponding structure, such as a suitably programmed processor and associated memory.

It should also be appreciated that particular combinations of the various features described and defined in any aspects of the invention can be implemented and/or supplied and/or used independently.

These and other aspects of the present invention will become apparent from the following exemplary embodiments that are described with reference to the following figures in which:

### BRIEF DESCRIPTION OF FIGURES

FIG. 1 shows an exemplary computer device on which the methods described herein may be implemented;

FIG. 2 shows an overview of an exemplary network of devices;

FIG. 3 shows an exemplary user interface display of a multi-feed session, comprising an ensemble of feeds, as transmitted across the network of devices;

FIG. 4a shows an exemplary user interface display during initialisation of a session;

FIG. 4b shows an exemplary user interface display of a multi-feed session during the process of adding an additional feed;

FIG. 5 shows a user interface display of the preview display while an obscuring shape is being applied;

FIG. 6 shows the preview display of the imaging feed of FIG. 5 while a further obscuring shape is being applied;

FIG. 7 shows an exemplary workflow of the interaction of a user with the user interface;

FIG. 8 shows an exemplary user interface display of a multi-feed session a multi-feed display to be edited;

FIG. 9 shows the preview display of an imaging feed as an obscuring shape is being edited;

FIG. 10 shows a logic flow of the construction of an imaging feed stream;

FIG. 11 shows a logic flow of the transformation of the imaging feed; and

FIG. 12 shows a visual illustration of the flow of the transformation of the imaging feed.

### SPECIFIC DESCRIPTION

### System Overview

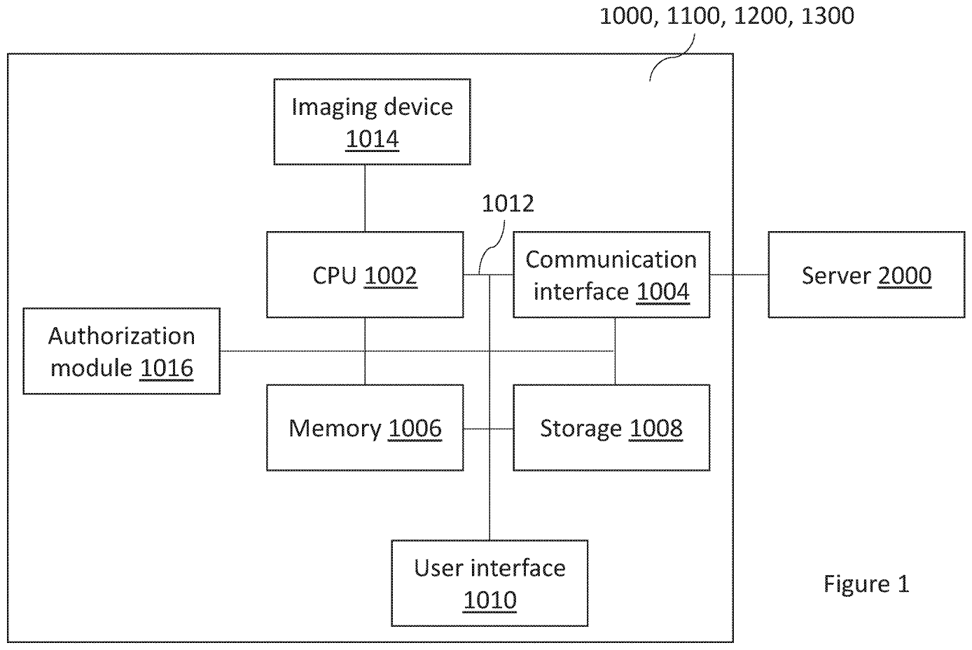

Referring to FIG. 1, the methods, apparatuses, and systems disclosed herein are typically implemented using at least one computer device 1000, 1100, 1200, 1300 connected to or connectable to a server 2000, and are typically computer-implemented methods.

The computer device 1000 comprises a processor in the form of a computer processing unit (CPU) 1002, a communication interface 1004, a memory 1006, storage 1008, a user interface 1010, and an authorization module 1016 and coupled to one another by a bus 1012. The CPU 1002 is further connected to an imaging device 1014, which is typically a video camera, but may be any form of device for generating an imaging feed (either real or computer-generated). For example, it may provide an imaging feed comprising visual test results, x-rays, computer-generated visualisations etc. Each computer device 1000, 1100, 1200, 1300 may comprise or be connected to more than one imaging device 1014.

The CPU 1002 executes instructions, including instructions stored in the memory 1006 and/or the storage 1008.

The communication interface 1004 enables the computer device 1000 to communicate with the server 2000 and with other computer devices. The communication interface 1004 may comprise an Ethernet network adaptor that couples the bus 1012 to an Ethernet socket. Such an Ethernet socket can be coupled to a network, such as the Internet. It will be appreciated that the communication interface may be arranged to communicate using a variety of mediums, such as area networks (e.g. the Internet), infrared, and Bluetooth®.

The memory 1006 stores instructions and other information for use by the CPU 1002. The memory is the main memory of the computer device 1000. It usually comprises both Random Access Memory (RAM) and Read Only Memory (ROM).

The storage 1008 provides mass storage for the computer device 1000. In different implementations, the storage is an integral storage device in the form of a hard disk device, a flash memory or some other similar solid state memory device, or an array of such devices.

The user interface 1010 enables users to interact with the computer device 1000 and comprises an input means and/or an output means. For example, the user interface may comprise a display and an input/output device, such as a keyboard, a mouse, or a touchscreen interface.

The authorization module 1016 defines and implements authorizations of the device 1000, 1100, 1200, 1300. This can define which entities, such as further devices and/or users, are authorized to provide instructions to be implemented by the computer device 1000, 1100, 1200, 1300. The ‘authorization module’ of each device preferably simply connotes an entity (processor and/or device) configured to perform authorizing functions for that device and/or means for authorizing for that device. This authorization module may be a module provided on each device, or it may be located at the server. It may be a module of a processor, a separate module or a module embodied in a processor. It may refer to any entity configured to perform authorizing instructions. Such instructions may be recorded in memory of a device and/or stored in a server and transmitted to the device.

The present invention can preferably be implemented on a generic computer device. This enables participants to participate in telesurgery sessions without requiring specialist hardware.

A computer program product is disclosed that includes instructions for carrying out aspects of the method(s) described below. The computer program product may be stored, at different stages, in any one of the memory 1006, the storage 1008 and/or a removable storage (e.g. a universal serial bus storage device). The storage of the computer program product is non-transitory, except when instructions included in the computer program product are being executed by the CPU 1002, in which case the instructions are sometimes stored temporarily in the CPU or memory. The instructions of the present invention are typically recorded at the server 2000. The server 2000 can preferably send the required instructions to the CPU 1002 via the communication interface 1016 to perform the method as described. The server 2000 may send updated or amended instructions.

It should also be noted that the removable storage is removable from the computer device 1000, such that the computer program product may be held separately from the computer device from time to time. Different computer program products, or different aspects of a single overall computer program product, are present on the computer devices used by any of the users.

There is also disclosed a system (e.g. a network) that comprises a plurality of computer devices, where each of these computer devices may carry out different parts of the methods disclosed herein. Each of the computer devices may be configured for a particular purpose. Typically, the plurality of computer devices is arranged to communicate with each other and/or the server 2000 so that data can be transmitted between the computer devices or between the devices and the server 2000. Each of the computer devices may be defined by different authorizations.

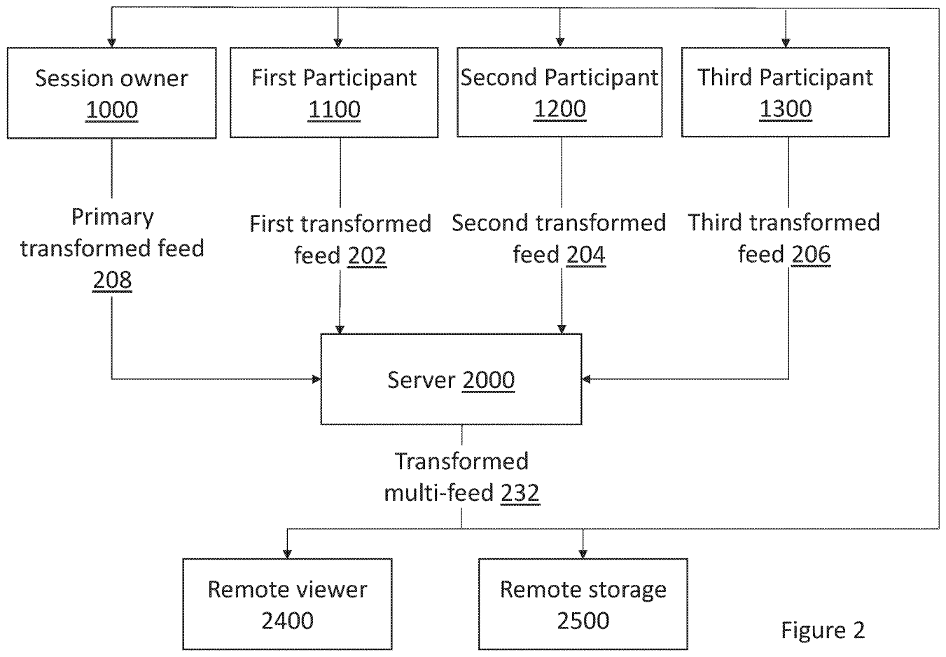

FIG. 2 shows a schematic overview of the devices which form a network that can be used to implement the present invention. The server 2000 transmits instructions to the further devices of the network to perform instructions. A system according to the present invention allows a first computer device, known as the session owner device 1000, to initiate a telesurgery session in which multiple imaging feeds can be collated and displayed, forming an ensemble display. The imaging feeds can originate from imaging devices of the session owner device 1000, a ‘first participant’ device 1100, ‘second participant’ device 1200, and/or ‘third participant’ device 1300. Each of these devices typically comprises the same component parts as the exemplary computer device illustrated in FIG. 1. In particular, each of the session owner device 1000, the ‘first participant’ 1100, ‘second participant’ 1200, and ‘third participant’ 1300 devices comprise an imaging device 1014 and a communication interface 1004 are configured to transmit imaging feeds from the imaging device 1014 to the server 2000. The imaging feeds may originate from physical imaging devices such as a computer camera, capture card or other imaging feed (such as a video) generating device. The imaging feeds may alternatively be user generated, for example via screen sharing, or a virtual camera. Typically, up to four imaging feeds can be active at any particular time.

The session owner device 1000, a ‘first participant’ device 1100, ‘second participant’ device 1200, and ‘third participant’ device 1300 may be provided in the same location or different locations (including some in the same location and some elsewhere) and may be provided in any combination. For example, the session owner device 1000 may be located in a surgeon's office in a hospital, the first participant device 1100 and second participant device 1200 located in the operating theatre of the hospital, and the third participant device 1300 located in a university in a different city.

The imaging feeds are transformed at the device from which they originate to generate a transformed imaging feed, in which regions are obscured (this is performed by a method which will be described later). This transformation is performed by the CPU 1002 on the device. The transformed feeds are then sent via the communication interface 1004 to the server 2000. The imaging feeds are not transmitted to the server 2000 in an untransformed state, nor are they recorded in an untransformed state. This enhances the privacy control of the imaging feeds.

The server 2000 collates the transformed feeds into a ‘masked’ multi-stream feed 232. The multi-stream feed 232 is distributed to any viewing devices of the session, including the session owner 1000 and participants 1100, 1200, 1300 and any other remote viewers 2400. These devices can view the transformed or ‘masked’ multi-stream feed via their user interfaces 1010. Continuing the example above, user interfaces of the device located in the surgeon's office, operating theatre and university will all display the multi-stream feed as transmitted by the server 2000. The remote viewer 2400 may be at any location in the world and may comprise multiple remote viewers in different locations all over the world. Typically, the remote viewers will require authorization to access the feed from the server. This may be an authorization associated with the user and/or the device.

The multi-stream feed 232 may also be recorded to a remote storage 2500 and/or to the internal storage 1008 of one or more of the devices. This means that the feed is only ever transmitted or saved with the obscured regions applied (i.e. the imaging feeds are only ever transmitted or saved in the ‘transformed’ state). As such, any viewer, whether in real time or via playback, only ever sees the imaging feed with the obscuration applied. As will be explained in further detail later, the imaging feed is transmitted and saved with the obscuration embedded within the file, such that it cannot be easily removed. This provides security that sensitive information cannot be revealed.

### User Interface Display of the Privacy Control Tool

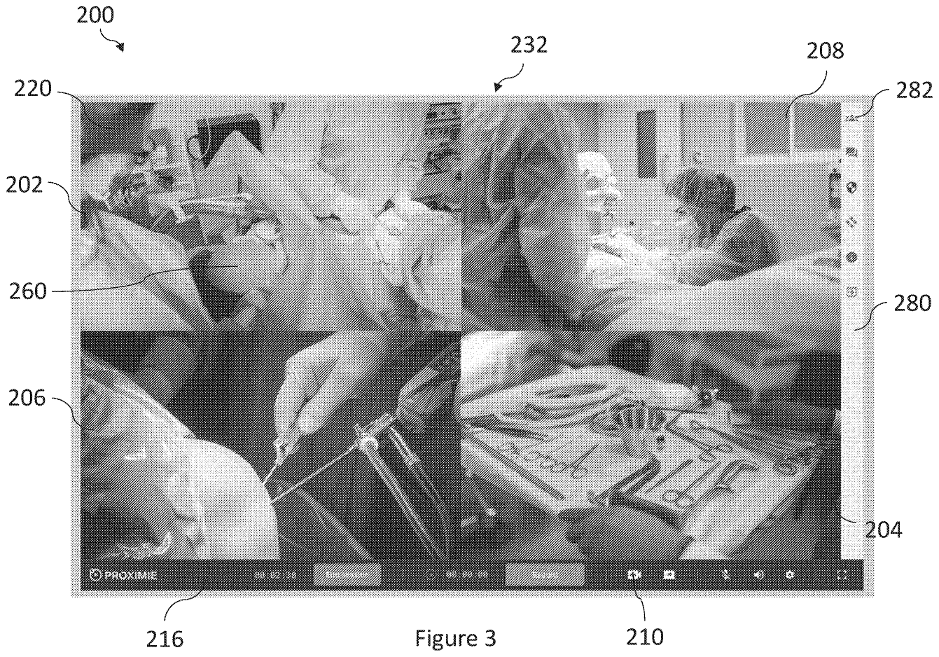

The server 2000 receives the imaging feeds from the devices 1000, 1100, 1200, 1300 and then collates them into an ensemble display, or ‘multi-feed’ display. The server 2000 distributes the multi-feed display to any viewing devices of the session, including the session owner 1000, participant devices 1100, 1200, 1300 and any further remote viewers 2400. FIG. 3 shows an exemplary embodiment of the user interface display 200 of the software according to the present invention. The user interface display 200 of FIG. 3 is in the multi-feed display 232 configuration, which displays four imaging feeds 202, 204, 206 and 208. The multi-feed display 232 is split into four quarters, each displaying one of the imaging feeds. In the present example, the four different imaging feeds show a video feed of an operating theatre from four different views: a primary video feed 208 shows the surgeon, a first video feed 202 shows the anaesthetist, a second video feed 204 shows close-up view of the operating theatre tool table, and a third video feed 206 shows a close-up view of the surgical site. Alternative imaging feeds may also be used, including combinations incorporating imaging feeds from a different geographical location.

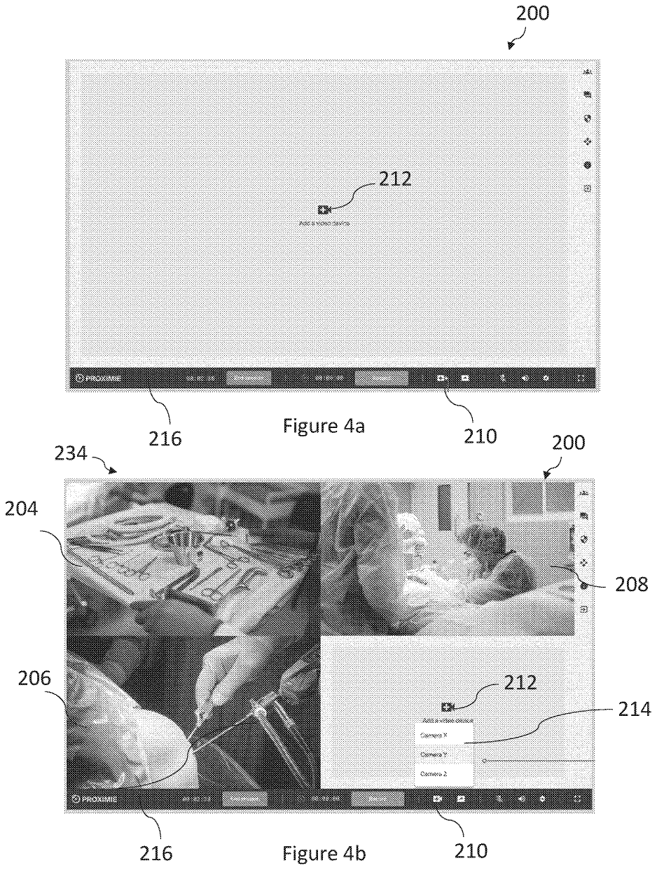

The session owner 1000 initiates a session, and the session owner 1000 and each of the participant devices 1100, 1200, 1300 can add imaging feeds. FIG. 4a shows how the user interface display 200 may appear before any imaging feeds have been added. The user can select the ‘Add a video device’ button 212 in the centre of the user interface display 200 or can select the ‘Add a video device’ button 210 in the tool bar 216. This can prompt the user interface to display a list of available imaging devices connected to that device, for example cameras, from which the user can choose to add an imaging feed.

FIG. 4b shows how the user interface display 200 may appear once some of the feeds have already been published to the session. In this case, the user interface display 200 shows the primary imaging feed 208, the second imaging feed 204, and the third imaging feed 206. These have already been added by, for example, the second participant 1200, the third participant 1300 and the session owner 1000. Three of the four quarters of the multi-feed display 232 are populated by these feeds. The fourth quarter displays an ‘Add a video device’ button 212, which the user can select to initiate the process of adding a new feed. Alternatively, the user can select the ‘Add a video device’ button 210 in the tool bar 216. Selection of either of the ‘Add a video device’ buttons 210, 212 may prompt the user interface to display a selectable list 214 of the available imaging devices. These are not limited only to video devices but may include any imaging device.

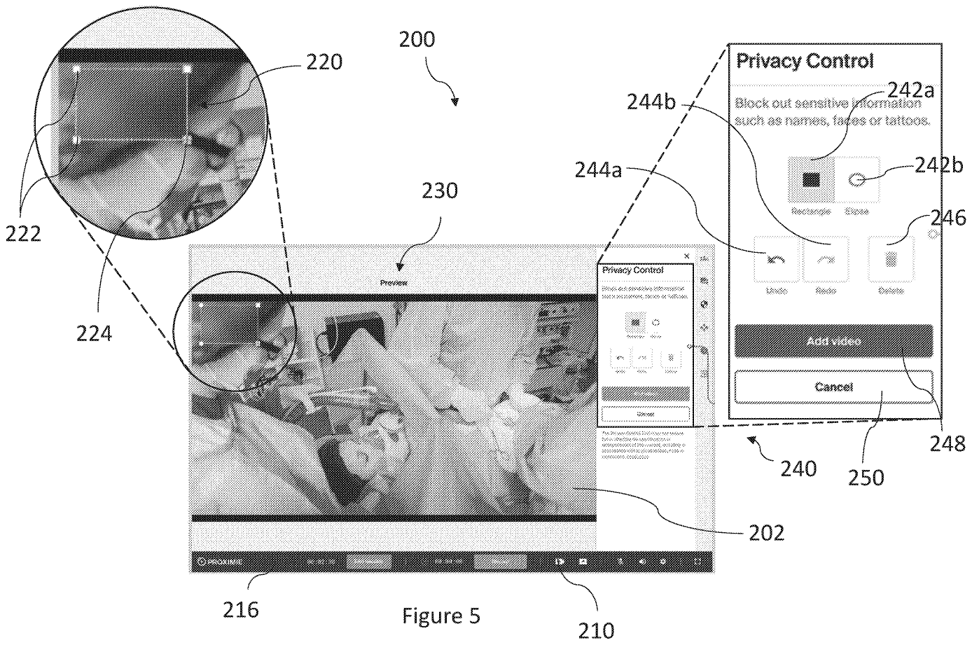

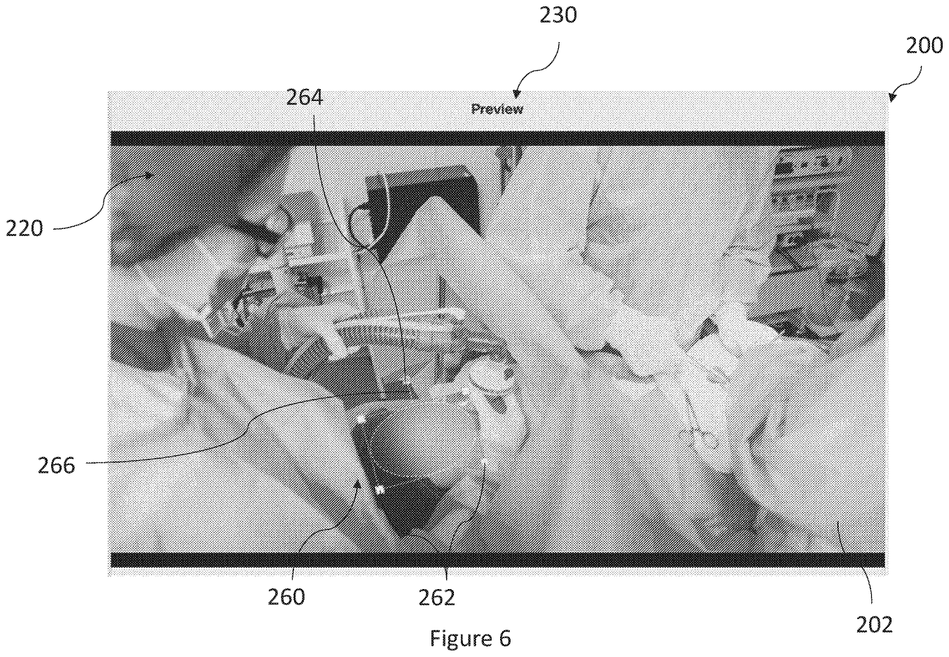

Upon selection of one of the cameras from the selectable list 214, the user interface 200 displays a preview display 230. This preview display 230 is shown in FIGS. 5 and 6. The preview display 230 shows the imaging feed from the chosen imaging device, which in this case is a video camera. The CPU 1002 at the relevant device performs instructions locally to display the preview display 230 on the local user interface 1010. These instructions may have been sent from the server 2000 and stored in the local memory 1006 of the relevant device (this storage may be temporary). The preview display 230 and the imaging feed are not transmitted to the server 2000 at this point. Before a user at the relevant device 1000, 1100, 1200, 1300 has confirmed that the imaging feed should be shared to the session, the imaging feed is visible only to the device 1000, 1100, 1200, 1300 from which the imaging feed originates. This allows sensitive information to be obscured from the imaging feed before it is transmitted or shared. This can be referred to as ‘privacy control’. The user interface display 200 comprises a privacy control toolbox 240, an enlarged representation of which is shown inset in FIG. 5.

In the exemplary implementation as illustrated, the privacy control toolbox 240 comprises selectable tools for adding obscuring shapes or ‘masking’ shapes. The user can select the shapes to be used, and define their location, size and/or orientation. This means the user can add and edit shapes to place over regions of an imaging feed which they would like to be obscured. The participant device implements the obscuring shapes by applying a ‘transformation’ to the imaging feed to generate a transformed imaging feed. (The details of how the transformation is implemented will be explained in detail in a later section.)

In particular, the privacy control toolbox 240 comprises a button for applying a rectangular mask 242a and a button for applying an elliptical mask 242b to the preview display 230. The privacy control toolbox 240 further comprises an ‘Undo’ button 244a, a ‘Redo’ button 244b, a ‘Delete’ button 246, an ‘Add video’ button 248 and a ‘Cancel’ button 250 for performing actions in the preview display 230. The buttons are typically displayed having a representative icon and/or text indicating their purpose. For example, the rectangular mask button 242a may display a rectangle icon, the elliptical mask button 242b may display an ellipse icon, the ‘Undo’ button 244a may show a left-pointing curved arrow icon, the ‘Redo’ button 244b may display a right-pointing curved arrow icon, and the ‘Delete’ button 246 may display a bin icon.

In FIG. 5, the rectangular mask is selected and so the relevant button 242a is shown in an ‘Active’ mode. The buttons of the privacy control toolbox 240 are typically provided in one of four states: ‘Disabled’, ‘Inactive’, ‘Hover’ and ‘Active’. In the ‘Disabled’ state, the button is not selectable, and is typically displayed with low colour contrast. For example, the main body of the button may be in white and the icon displayed in a pale grey. The ‘Undo’ button 244a is typically provided in the disabled state until a change has been made. Once a change has been made, the ‘Undo’ button 244a may then move to the ‘Inactive’ state. In the ‘Inactive’ state, a button is selectable but has not been selected. In this state, the button will typically be displayed with greater colour contrast. For example, the body of the button may remain white and the icon displayed in a darker grey. If the user then hovers the cursor over that button, it moves from the ‘Inactive’ state to the ‘Hover’ state. In this state, the button may be displayed as ‘highlighted’ in that the intensity of both the background and the icon is increased. For example, the background may be provided as a pale grey and the icon as a darker shade of grey or black. If the user then selects the button (for example the rectangular mask button 242a and the elliptical mask button 242b), it then moves to the ‘Active’ mode. For example, when the rectangular mask button 242a is in the ‘Active’ mode, the user can then draw a rectangular mask on the video stream. The ‘Active’ mode may be indicated by the icon shape becoming filled. The button may also display a colour, for example the shape buttons (rectangular 242a and ellipse 242b) may be displayed with low intensity blue background and the icon in a higher intensity blue. The ‘Undo’ 244a and ‘Redo’ 244b buttons may remain in grey scale and the ‘Delete’ button 246 may be displayed in red (which can act as warning that a shape is about to be discarded). The rectangular mask button 242a and the elliptical mask button 242b cannot be selected at the same time, but rather the user can toggle between them. The ‘Undo’ button 244a, ‘Redo’ button 244b and ‘Delete’ button 246 are not activated for an extended period of time but are activated momentarily when selected to perform a discrete action. When the rectangular mask button 242a or the elliptical mask button 242b are activated, the cursor display changes from a selection arrow to a cross, indicating that the drawing mode of the relevant shape is activated. In some implementations, other means of interacting with the user interface display 200 may be used, for example keyboard shortcuts may also or alternatively be enabled to allow a user to perform the relevant actions.

A further inset in FIG. 5 shows the area in which a rectangular mask 220 is being applied to the imaging feed 202. In this example, the top left corner of the imaging feed 202 displays personal details about the patient which a user wishes to obscure. As such, a rectangular mask 220 is drawn in this location, causing the regions defined by the shape to be blurred and thereby obscure the information. While the rectangular mask button 242a is selected, the user can click and drag to draw the shape. In this state, the cursor 224 is displayed as a cross to indicate the drawing mode is activated. In the preview display 230, an outline is provided while the shape is selected to clarify the location and shape on the imaging feed 202. In the illustrated embodiment, this outline is implemented as a dashed white line. Once the user creates the shape and releases the mouse click, the shape remains active and selected, displaying the borders, rotation handle (not shown when the shape is being drawn) and the resizing handles 222. At this point, however, the rectangular mask button 242a on the privacy control toolbox 240 transitions to the ‘Inactive’ state. This allows a user to click away from the shape to deselect it. The cursor display changes back to the arrow display. When the shape is deselected, it appears as it will in the live feed, allowing a user to preview how the shape will look in the session (for example, without a border). The shape can be selected once again by clicking on it, or by clicking and dragging a selection rectangle which encompasses the shape. More than one shape can be selected via either method. In this mode, the cursor is shown as an arrow to indicate the selection mode.

The rectangular mask 220 can be resized by selecting one of the resizing handles 222, typically provided at the corners of the shape and at the centre of each of the edges. A user can resize a shape by dragging a resizing handle 222 to a new position in order to enlarge or reduce the rectangle. The rectangular mask 220 can also be rotated by selecting an end of a rotation handle, which extends from the centre of one edge of the rectangle 220 (this is not shown in FIG. 5 as the rotation handle is not displayed while the user is drawing the shape). The user can change the rotation by selecting the rotation handle and dragging it until the shape is rotated to a desired orientation. Additionally, while the shape is selected, the user can drag the whole shape to a new location to move it. During such an action, the cursor is displayed as an arrow cross.

More than one region of the imaging feed 202 may be obscured at any time. FIG. 6 shows the imaging feed 202 display of the user interface 200 in the preview display mode 230 while a further shape is being applied. In this instance, an elliptical mask 260 has been chosen by the user. This is done by selecting the elliptical mask button 242b. The elliptical mask 260 can be resized and reshaped by a user upon selecting a corner 262 and dragging the corner until the corner is moved to a position such that a desired shape and size has been achieved. The shape can be rotated by the cursor 264 selecting the rotation handle 266. The rotation handle 266 comprises a line extending perpendicularly from one side of the ellipse with a selectable handle provided at its distal end. The rotation handle 266 can be moved by a user until the desired orientation of the shape 260 has been achieved. In the example as shown in FIG. 6, the elliptical mask 260 has been resized, rotated and positioned such that the patient's face is obscured. In this manner, the surgeon can enhance the privacy of the patient.

The privacy control toolbox 240 further comprises an ‘Undo’ button 244a, and a ‘Redo’ button 244b. The ‘Undo’ button 244a is disabled until a user has made a change (for example, an edit to the size or positioning of a shape). After the user has made a change, the ‘Undo’ button 244a is enabled, and can be selected by the user to undo that change. At this point, the ‘Redo’ button 244b can become enabled. If selected by a user, it can cause the action to be re-performed.

The privacy control toolbox 240 further comprises a ‘Delete’ button 246, which can be used to delete a selected shape or shapes from the preview session. Any shape can be reconfigured during the preview session by clicking the cursor over it to select it. When a shape is selected, the ‘Delete’ button moves from the ‘Disabled’ state to the ‘Inactive’ state (or ‘Hover’ state or ‘Active’ state, in dependence on where the user places and clicks the cursor). When no shapes are selected, the ‘Delete’ button moves to the ‘Disabled’state.

The shapes as defined by the user in the user interface display 200 define the regions which should be obscured. The shapes therefore define the transformation which will be applied to the imaging feed to generate the transformed imaging feed.

Once the user is satisfied with the shapes applied to the imaging feed, they can elect to publish the transformed version of the imaging feed to the session by selecting the ‘Add video’ button 248. This causes the transformed imaging feed to be transmitted to the server 2000. The server 2000 then broadcasts the transformed imaging feed as part of the session, such that it is viewable by all viewing devices of the session (i.e. the session owner 1000, the participant devices 1100, 1200, 1300 and remote viewers 2400).

The transformation of each imaging feed is performed locally at the participant device from which the imaging feed originates. The transformed imaging feed is then sent to the server 2000, as shown in FIG. 2. The server 2000 collates the transformed feeds into a transformed multi-feed broadcast, which it distributes. The collated imaging feeds of the session are transmitted from the server 2000 as a transformed multi-feed display 232 to each of the session owner 1000, the participant devices 1100, 1200, 1300, any remote viewer devices 2400, and any remote storage 2500.

The untransformed imaging feed is only viewable at the relevant participant device from which it originates, and is not transmitted beyond it, nor is it recorded. Therefore, the imaging feed is only ever transmitted in the transformed state, in which any sensitive information can be obscured from view by the obscuring shapes. Neither the server 2000 nor the session owner 1000 receive an untransformed version of the imaging feed. Therefore, the unobscured imaging feed cannot be recovered and so the obscuration is immutable. This improves the security of the privacy control tool.

In some implementations, when the user selects the ‘Add video’ button 248 after applying obscuring shapes, an alert or warning may be output recommending instructing participants not to pan, tilt or zoom the imaging devices. For example, this may read: “PTZ camera—We recommend instructing participants to not pan, tilt or zoom cameras that have privacy masks applied.” This is because the region the user wishes to obscure may move to a different location on the display when any of these functions are applied to the imaging feed, causing them to no longer be obscured by the shapes. This warning may be output in dependence on the system detecting that the imaging device is a PTZ camera or other imaging device with PTZ functionality, or it may be output as a default. This alert may also be output in dependence on the system detecting that the user is about to control the PTZ camera (or imaging device) while the feed has a transformation defined.

The privacy control toolbox 240 further comprises a ‘Cancel’ button 250, which cancels the editing in the preview session, and causes the user interface display 200 to exit the preview display 230.

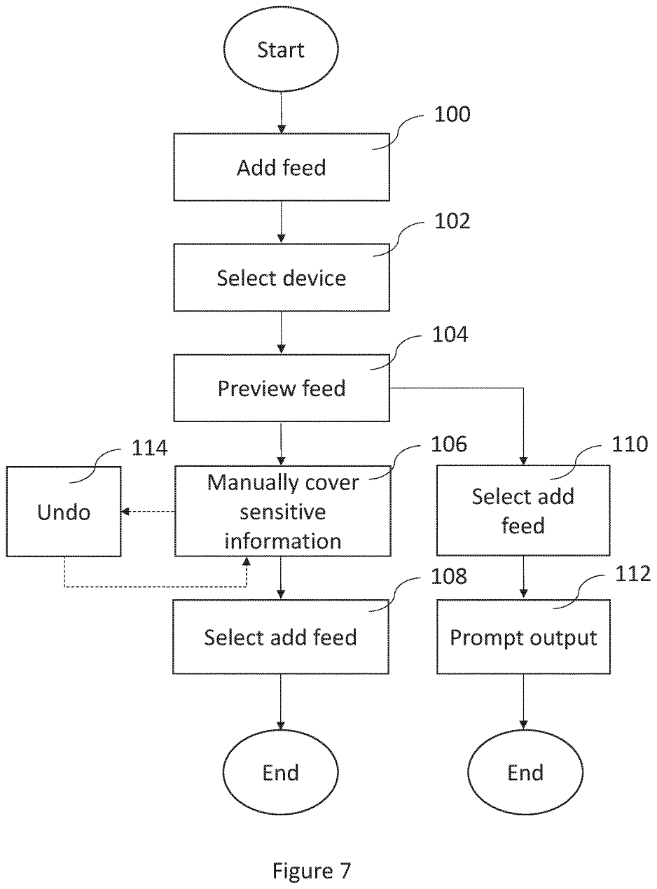

FIG. 7 shows an example workflow of interactions of the user with the user interface display 200 of one of the devices 1000, 1100, 1200, 1300. At step 100, the user can add an imaging feed by selecting the ‘Add a video device’ button 212 on the centre of the screen, or by selecting the ‘Add a video device’ button 210 in the tool bar 216. The system may then output the selectable list 214 of the available imaging devices, from which a user can then select a device in step 102. This prompts the system to display the preview display 230, at step 104. Within this preview display 230, the user can manually cover any sensitive information at step 106 by the processes described above. Any changes can be ‘undone’ at step 114 and obscuring shapes can be added and manipulated further, in an iterative process. Once the user is satisfied with the configuration of the obscuring shapes applied, they can then select to add the imaging feed to the session at step 108. Alternatively, the user can select to add the imaging feed directly to the session at step 110. If the user selects to add the imaging feed without adding any obscuring shapes, the system will output an alert or prompt to the user to check for any sensitive information at step 112. This may, for example, include personally identifiable information (PII). Typically, the user will need to confirm that they wish to proceed before the imaging feed is published to the session.

### Editing Privacy Control Masks and Authorizations

After the imaging feeds have been transmitted to the session, the server 2000 transmits the multi-feed display 232 to the viewing devices, typically comprising the session owner 1000, each of the participants 1100, 1200, 1300 and any further remote viewing devices 2400. This shows the four transformed imaging feeds 202, 204, 206, 208 side-by-side, for example in quarters, as shown in FIG. 3. These transformed imaging feeds will include the obscuring shapes as applied when the user added the feed, and as described above. There may, however, be instances when it is desirable or necessary to edit the shapes during a session. This may be because the patient has moved or the imaging device has been moved or reconfigured (for example by effecting changes to pan, tilt, or zoom settings). These changes can cause regions considered to contain sensitive information to be no longer obscured.

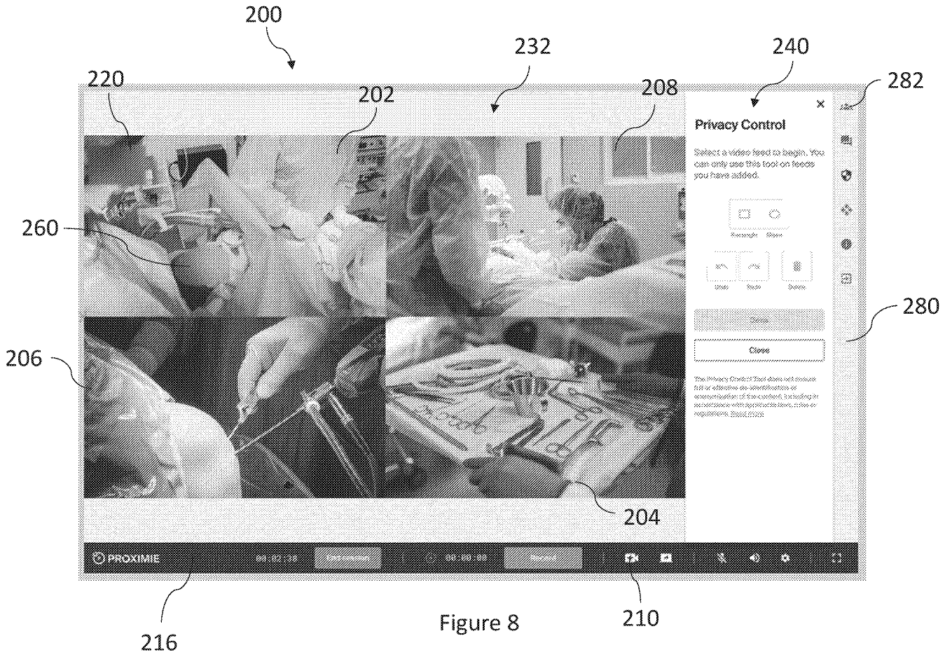

The user can select the privacy control icon 282 from the side navigation bar 280 of the user interface display 200. This will cause the privacy control toolbox 240 to be displayed, as shown in FIG. 8. A user can then select one of the imaging feeds to edit, typically by hovering over the feed and clicking to select it. A feed may only be edited by the participant device which added it, and from where the feed originates, or by the session owner 1000. For example, if the first video feed 202 is transmitted from the first participant 1100 device, then only the first participant 1100 or the session owner 1000 can edit the privacy control settings (the obscuring shapes). The second participant 1200 and third participant 1300 cannot edit the first feed 202. If the second feed 204 was added by (and originates from) the second participant device 1200, then only the second participant 1200 or the session owner 1000 can edit the transformation applied to it (i.e. by editing the obscuring shapes). The first participant 1100 and third participant 1300 cannot edit the second feed 204. This allows the session owner 1000 to edit the transformation of each feed. This can be advantageous, for example if the shapes need to be moved but one or more of the users at the participant devices 1100, 1200, 1300 is busy (for example, a surgeon may be scrubbed in). However, it does not allow other participants to edit the transformations, as these edits could reveal regions the original participant wished to obscure (which may contain sensitive information).

The authorization module 1016 for each of the computer devices (the session owner 1000 and the participant devices 1100, 1200, 1300) defines which of the other devices is authorized to control the imaging feed originating from that device. For example, the authorization module 1016 of the session owner device 1000 defines that only the session owner 1000 is authorized to control the primary imaging feed 208 (which originates from the imaging device 1014 of the session owner 1000). The authorization module of the first participant device 1100 defines that the first participant device 1100 is authorized to control the first imaging feed 202 (including viewing it in an untransformed state) and the session owner 1000 is authorized to edit the transformation. The authorization modules 1016 of the second participant device 1200 and third participant device 1300 define similar authorizations that the session owner 1000 is authorized to make changes to the transformation of the second feed 204 or third feed 206 (respectively), but only that same device (the second participant device 1200 and third participant device 1300, respectively) is authorized to view the untransformed imaging feed.

If a participant device is not authorized to edit a particular feed, the privacy control toolbox 240 will not become active on the user interface display 200 of that participant device. This may occur if, for example, the second participant 1200 attempts to the edit the obscuring shapes on the first video feed 202 (which originates from the first participant 1100). The system may output a message “You do not have permission to modify this feed” (or similar message).

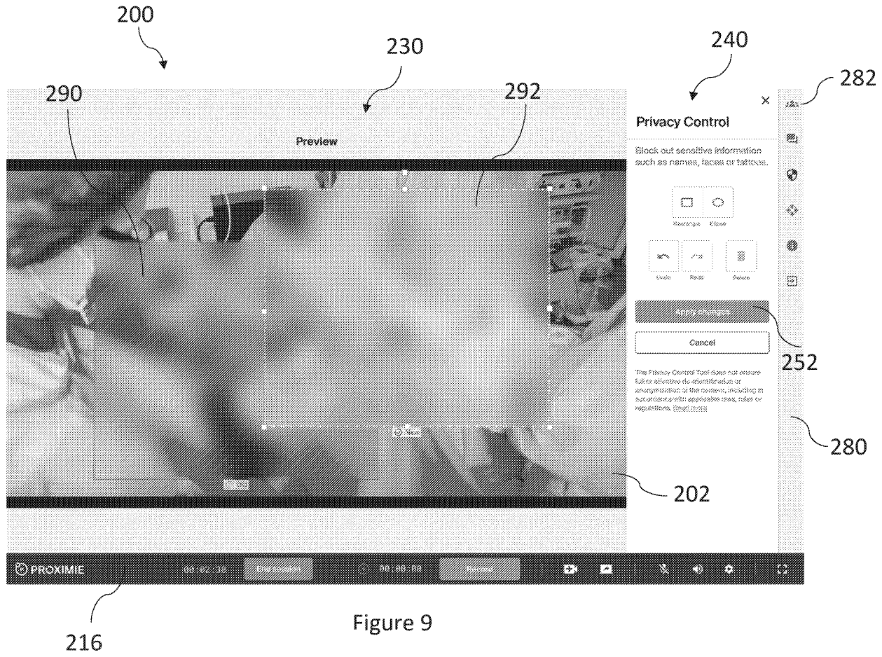

If the participant device is authorized to edit a feed, the user interface display 200 enters the preview mode 230 and the privacy control toolbox 240 becomes active. This is shown in FIG. 9. The editing controls and methodology are the same as for the preview mode 230 describe previously in respect of when a user is adding the imaging feed. The user can use the same commands to resize, rotate, add, delete and move the obscuring shapes.

The imaging feed is transmitted from the participant device 1100 to the server 2000 only in the transformed state and is broadcast from the server 2000 to the session owner 1000 only in the transformed state. This means that if the session owner 1000 enters the preview mode 230 of the first feed 202 and performs an edit to move an obscuring shape, the un-obscured imaging feed in that region is unavailable. Therefore, as shown in FIG. 9, the ‘old’ position 290 of the obscuring shape remains obscured (in this case, blurred). The user interface display 200 may indicate this old position 290 by displaying indicative markers in this region. For example, as illustrated in FIG. 9, a border and hatching may be applied to the old position 290. The new, editable position 292 of the shape will typically show a preview of the obscuration effect (in this case blurring). A different border may be applied to the new position indicating that it is currently selected and editable (for example, a dotted white line, as is also used in the preview mode when a user is adding an imaging feed). The old position 290 and the new position 292 may also be labelled accordingly.

The editing in the preview mode 230 is not published to the session until the user selects the ‘Apply changes’ button 252 on the privacy control toolbox 240. Therefore, typically, none of the other devices will be able to see the changes being made until they are finalised. Typically, the preview display 230 may never be transmitted beyond the device on which it is shown. For example, if the session owner 1000 enters the preview display 230 and makes changes to the obscuring shapes, these amendments are made locally. The relevant participant device 1110 does not view the changes as they are being made. Only when the changes are finalized will the data be transmitted to the server 2000, and then broadcast to the relevant participant device 1100. In some implementations, a notification may be transmitted to the relevant participant device 1100 that changes are being made, optionally in dependence on the participant device 1100 attempting to enter the preview mode. When a user attempts to enter the preview display 230 to amend the shapes, the system may output a warning that changes are being made on another device. In some implementations, the system may block the user from entering the preview display 230 and/or from making changes at that time.

The transformation of imaging video feed is applied at the relevant participant device, and then sent to the server 2000 with the mask applied. Once the user clicks ‘Apply changes’ 252, even if the user is at the session owner device 1000, the transformation is amended at the participant device 1100. In the case that the shapes have been defined and/or edited at the session owner 1000, the new, updated transformation (as defined by the new configuration of the obscuring shapes) is transmitted from the session owner 1000 via the server 2000 to the relevant participant device 1100. The updated transformation is typically transmitted as a set of amended computer-implementable instructions. These instructions typically comprise a list (array) of shapes (i.e. the obscuring shapes) where each shape has a coordinate position (for example, an xy coordinate), rotation angle, shape type, and shape dimensions (width and height for a rectangle; or radii, Rx and Ry, for an ellipse). These instructions may be received by the participant device via the communication interface 1004 and stored in the memory 1006 and/or storage 1008 of the computer device. The CPU 1002 of the participant device 1100 then performs the new, amended instructions to apply the new, amended transformation. The transformed imaging feed 202, now comprising amended obscured regions, is transmitted from the participant device 1100 to the server 2000, where it is collated into the multi-feed display 232. Accordingly, only a transformed imaging feed 202 is transmitted beyond the participant device 1100.

### Video Feed Logic Flows

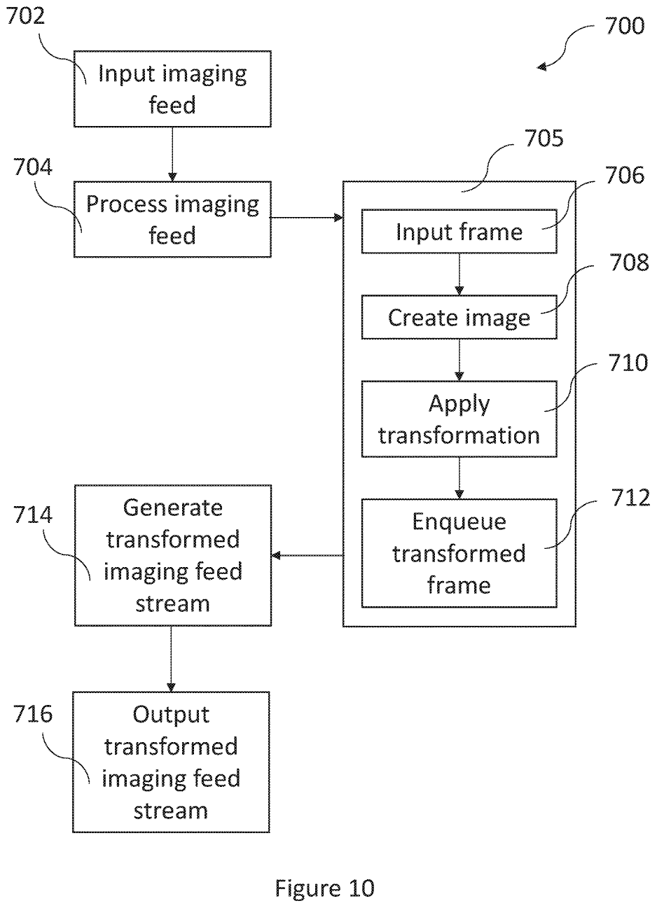

FIG. 10 shows an exemplary flow diagram of the logic flow associated with an imaging feed stream constructor 700. In this exemplary embodiment, the imaging feed is formed of a series of frames, which are imaged individually. Each resulting image is then transformed, and the series of transformed images are assembled to generate a transformed imaging feed stream. At step 702, an imaging feed, such as a video stream, is input from the imaging device 1014 to the CPU 1002, which performs the method processing steps as described according to instructions stored within the memory 1006 or storage 1008, and/or as transmitted via the communication interface 1004. At step 704, the CPU processes the imaging feed and inputs it into to a transformation stream 705. Typically, a media stream track processor may be used for piping the imaging feed on a frame-by-frame basis through a transformation function in order to apply the obscuring shapes as required.

During the transformation stream 705, each frame of the imaging feed stream is input at 706 and an image of that frame is created at 708 (for example, as a bitmap file). The image is transformed such that the regions indicated by the user are obscured at step 710. A new transformed frame, in which the relevant regions are obscured, is enqueued at 712. At step 714, the frames are assembled into an imaging feed stream or ‘track’. Typically, this is performed by a media stream track generator, which can enqueue the series of transformed frames according to their original timestamps. At step 716, a new, transformed imaging feed stream is output. It is this transformed imaging feed that is output from the CPU 1002 of the participant device, via the communication interface 1004, to the server 2000 to add to the session, as illustrated in FIG. 3.

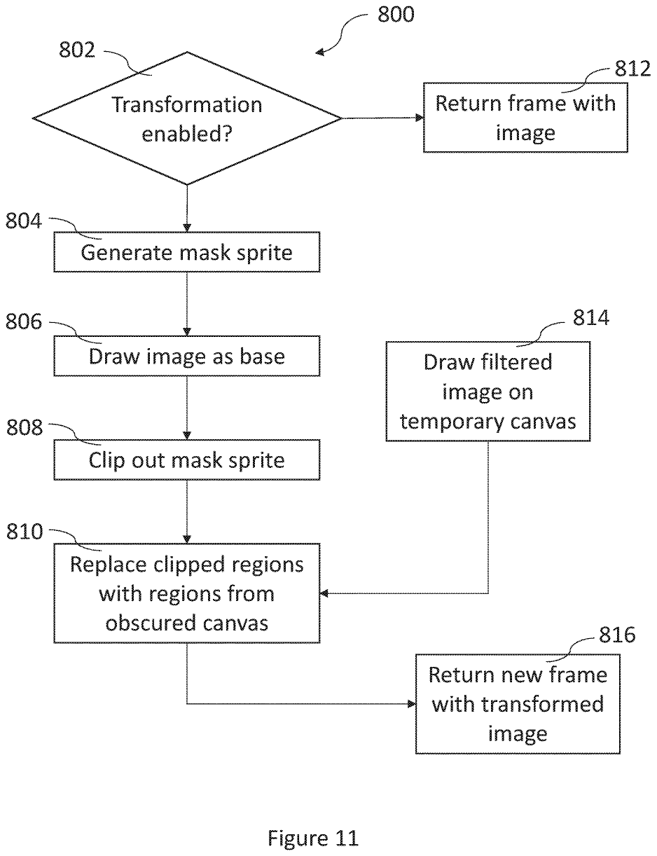

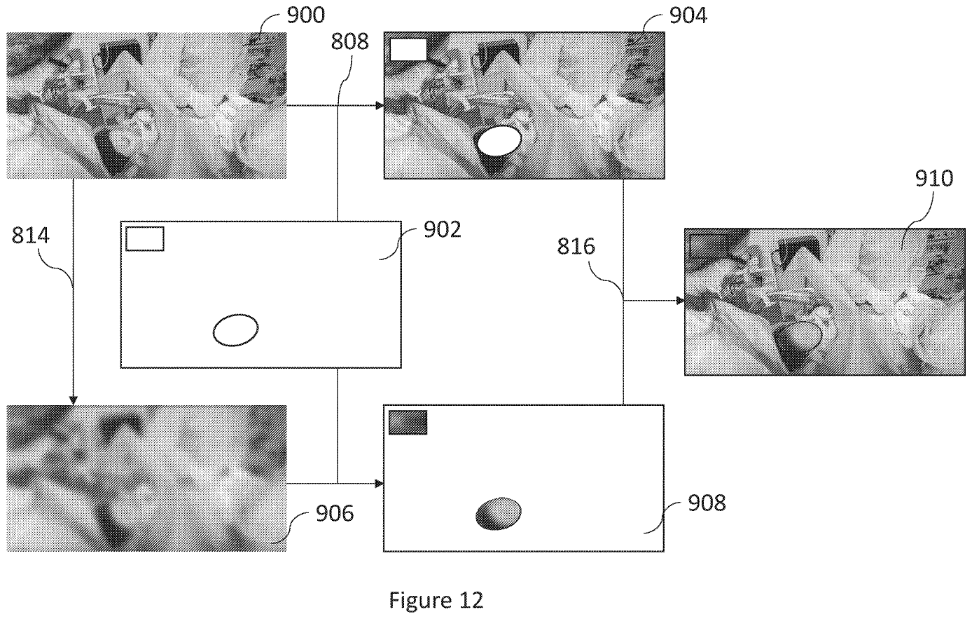

FIG. 11 shows a more detailed flow diagram of the logic flow of the transformation 800. The transformation 800 may be performed at the transformation step 710 of the constructor logic flow, as shown in FIG. 10. FIG. 12 shows an illustrative visualisation of images of the feed as created throughout the transformation flow. As shown in FIG. 12, first an untransformed image 900 of the feed is input. At step 802, the system first determines whether transformation is enabled (i.e. whether any obscuring shapes should be applied). If transformation is not enabled, the processor 1002 simply outputs a new frame from the image (step 812) and exits the logic flow. If transformation is enabled, the processor 1002 progresses to the next step of the logic flow. The processor 1002 may check if the frame has a width and height defined and if it does not, creates a new video frame using the last known frame.

At step 804, the CPU 1002 generates a mask sprite 902. A mask sprite 902 is a single set of instructions comprising the configuration of all the obscuring shapes, as shown in FIG. 12. The mask sprite 902 comprises a blank, transparent image or canvas of the same size as the original video frame, with all the shapes drawn over it in their proper position with solid colour filling (this can be any colour, but is typically white). When applying further instructions, these regions defining the shapes will thereafter become the ‘active areas’ affecting the output. The mask sprite 902 is constructed and defined according to the arrangement of obscuring shapes added and configured by the user in the preview mode 230 of the user interface 200. The mask sprite can therefore be considered to define the transformation. The mask sprite 902 has the same dimensions as the image of the frame. Processing efficiency is optimized by the use of a single mask sprite 902, which contains all of the obscuring shapes, rather than multiple smaller masks. This is because the mask sprite 902 is calculated once, only when obscuring shapes are changed (for example, moved, scaled, deleted, and/or added), and then is used to apply the masking instructions in one pass per frame without requiring re-calculation of individual sprite masks. This can greatly improve computational efficiency in comparison to looping over each separate obscuring shape on every frame and applying the masking instructions multiple times.

The system may not need to generate a new mask sprite 902 for each frame, if the configuration of the obscuring shapes has not been changed. Instead, the mask sprite 902 may be saved in the memory 1006 of the device, and the CPU 1002 recalls it when required. The CPU 1002 may advantageously first check that the dimensions of the image 900 are still the same as those of the mask sprite 902. The CPU 1002 may amend scale the dimensions of the mask spite 902 to those of the image 900 in consequence of determining a difference in their dimensions.

At step 806, the system draws the image as a base of an output canvas frame (this will also write over any old images which may be present, for example from a previous run of the transformation logic flow 800, for the previous frame).

At step 808, the mask sprite 902 is applied to the output canvas, resulting in the regions defined by the obscuring shapes being clipped (i.e. completely removed) from the image. The resulting image 904 comprises the image with the regions removed, as shown in FIG. 12.

At step 814, parallel to the process steps described above, a temporary canvas is created, onto which the image is applied. The CPU 1002 applies a filter effect to the temporary canvas, which has the effect of achieving an obscuration of the image. This is typically a blurring filter, resulting in a completely blurred video frame image 906 on the temporary canvas (i.e. the whole image is blurred, as shown in FIG. 12). The filter effect used is typically a gaussian blur filter. The value of the standard deviation of the gaussian blur function is chosen to maintain a compromise between achieving sufficient obscuring of information and minimizing the computational cost. This is typically chosen to be 25 pixels for a 720p video feed (i.e. a high-definition (HD) display with a resolution of 1280×720 pixels). However, the standard deviation may also be any value greater than 20 pixels, and preferably between 20 pixels and 45 pixels, more preferably between 20 pixels and 35 pixels, and even more preferably between 20 pixels and 30 pixels. The standard deviation values used will be different for imaging feeds of different resolution. Typically, however, the same ratio of standard deviation to resolution will be used (i.e. typically 25:720, but preferably between 20:720 and 45:720, more preferably between 20:720 and 35:720, and even more preferably between 20:720 and 30:720). For example, a standard deviation of 12.5 pixels is used for a 360p video feed. This can aid in maintaining a similar blurring effect across imaging feeds of different resolutions.

At step 810, corresponding regions of the blurred temporary canvas 906 are applied to the output canvas, to replace those regions which were clipped out by the application of the mask sprite in step 808. This means that for those regions, as defined by the mask sprite, the corresponding pixels (i.e. those at the same coordinates) of the blurred temporary canvas (shown as image 908 of FIG. 12), completely replace the removed regions of the original image. The resulting image 910 is therefore a ‘patchwork’ of the original image of the frame with the regions defined by the obscuring shapes removed 904, and corresponding regions of a blurred version of the original image 908. The position, shape and orientation of those regions is determined by the sprite mask 902, which is defined by the shapes applied and edited by a user or users via the privacy control toolbox 240. (Please note that FIG. 12 shows a border around the obscured regions of the transformed image 910 for emphasis, but these may not be present in the image as output.) In the exemplary embodiment, a gaussian blur filter is applied to a copy of the image on a temporary canvas. A gaussian blur achieves a blurring effect by convolving the image with a gaussian function. The gaussian function is defined by a standard deviation value (for example, 25 pixels). Therefore the blurring is dependent on the weighted average of neighbouring pixels. Typically, if a gaussian blur is applied only to a clipped region of an image, this can result in a reduced quality of blur at the outer edges of the region as the radius of the function extends out of the bounds of the region. By contrast, the method of the present invention can advantageously produce a more uniform blur effect across the obscured region inserted into the imaging feed. This is because the blur function is applied uniformly across the image, and then only the required portions (the ‘regions’) are extracted and output to the resulting image 910. This can facilitate the effective use of smaller regions, which might otherwise be severely compromised due to the poor blurring at their edges.

The method of the present invention also means that the ‘masked’ regions are comprised only of a blurred image, rather than a blurring mask being applied on top of sensitive information. As such, the imaging feed is not so much ‘masked’ as transformed. This transformation comprises completely replacing regions with corresponding regions of an alternative image. This means that the obscuration is an inherent part of the resulting image and cannot be removed later. The sensitive parts of the image, which a user wishes to obscure, are completely removed and replaced with corresponding regions of an alternative image (e.g. an obscured image). The obscuration is, as such, immutable.

Finally, at step 816, the resulting image 910 is output to form the new, transformed frame. The frames are assembled by a media stream track generator at step 714 to output the transformed imaging feed. The device 1000, 1100, 1200, 1300 then sends this transformed imaging feed 208, 202, 204, 206 to the server 2000.

The transformations take place simultaneously with the imaging feed, so that the video frames are transformed in real time. Typically, there is a processing lag time of no more than 500 milliseconds (ms), preferably no more than 300 ms, and most preferably no more than 200 ms. This lag time encompasses the time between the imaging device 1014 generating the feed and the communication device 1004 transmitting the transformed imaging feed to the server 2000.

If a user edits the obscuring shapes via the method outlined previously, this has the effect of changing the sprite mask 902 (i.e. changing the transformation) as soon as the user has ‘published’ their changes to the session (by selecting once the ‘Apply changes’ button 252, as shown in FIG. 9). This means all subsequent frames are transformed using the updated sprite mask 902, so that the obscuring shapes are located in the new configuration. This transformation is performed at the relevant participant device. For example, if the session owner 1000 changes the position of the mask applied to the first video feed 202, those changes to the sprite mask are implemented at the first participant device 1100. In this case, the session owner device 1000 sends instructions relating to the amended sprite mask (i.e. the amended transformation) via the server 2000 to the first participant device 1100. The first participant device 1100 stores the new instructions defining the amended sprite mask 902 in its memory 1006. The CPU 1002 of the first participant device 1100 from then on performs the new instructions to perform the processing steps as outlined above using the new sprite mask 902. The first participant device 1100 outputs the transformed imaging feed 202 to the server 2000, which may then broadcast it on to the session owner 1000 (among other devices). Accordingly, even though the session owner 1000 implements the changes to the sprite mask 902, it still does not view or receive an untransformed version of the imaging feed. (Unless, of course, the user deletes all of the obscuring shapes, in which case all participants and viewers would also see an untransformed imaging feed).

### Alternative Obscuration Techniques

In some embodiments, the obscuration may be implemented by applying a different effect to an alternative image. This may include applying a different filter to a copy of the image. For example, instead of application of a gaussian blur filter, the processor may apply a pixelating filter to the image on the temporary canvas.

In some simplified embodiments, the imaging feed may not be imaged at all. Instead, the processor may apply a solid colour to the temporary canvas. The clipped regions of the image (corresponding to the shapes) may then simply be replaced by corresponding regions of the solid colour. The colour may be predefined (for example, grey), or may be an average colour based on the surrounding pixels of the image or of the image as a whole. Nonetheless, these regions are completely replaced in the image (via a ‘patchwork’ technique as described above), rather than a mask being applied directly to (on top of) the image. This means the masking of the feed is immutable, which improves the security of the privacy control tool.

The method of obscuration may be chosen in dependence on the performance of the relevant participant device. This performance may be related to and/or defined by processing and/or connection speed (which may include upload and/or download speeds). The performance may be further defined using metrics comprising measured parameters, and these parameters may be measured continuously or intermittently. Standard metrics and methodology, as are known in the art, may be used to achieve this. For example, metrics may comprise the time the page takes to process 1 video frame (which can be extracted from WebRTC statistics). Further metrics may include page render frames per second (fps), which defines how many times the page is rendered per second. This may ideally be 60 fps on standard screens, and a lower value can be taken to indicate worse performance. Page render time per frame (TPF) may also be used, which defines the time it takes the page to fully render on each render cycle. This may ideally be 17 ms per frame for a standard 60 fps screen, but a higher value can be taken to indicate a worse performance. Additionally, the system may use as a metric the ratio of video stream frames processed (encoded/decoded) to video frames from source (camera or received over network). Ideally the process rate may be 100% (or very close to) and a lower value may be taken to indicate a worse performance. These are exemplary metrics only and may be used in any combination. Any further indicators deemed relevant to the overall performance of system may also be used.

The obscuration method may be determined in dependence on the values of certain parameters being measured or calculated to be above or below pre-set thresholds and/or within particular ranges. For example, if a metric falls below a first threshold value, the CPU 1002 performs a set of instructions to apply a pixelation filter to the alternative image instead of a blurring filter. If the metric falls even lower, to below a second threshold value, the CPU 1002 may perform a set of instructions whereby an alternative image formed of a solid colour is used to replace the regions, and the process does not comprise applying a filter to an image at all. The obscuration method may be varied in real time as determined and/or measured performance metrics and parameters vary. This can prevent disruptions to stability of the imaging feeds caused by the computational cost of applying the transformation. Different obscuration methods may thus be implemented at different participant devices of a session.

### Further Alternatives and Modifications

Various other modifications will be apparent to those skilled in the art. For example, while the detailed description provides an example use in a telesurgery system, it should be understood that the invention can be implemented in any situation where sensitive information may be transmitted via imaging feeds, and a user may wish to obscure that information securely and locally.

The imaging feeds as described in the present invention may encompass any type of imaging. These may be static imaging feeds or real-time imaging, such a video feed. The imaging feeds are not limited only to video photography but could also include other imaging techniques such as ultrasound. The imaging feeds may also encompass computer-generated 2D or 3D modelling visualisations.

The imaging feed stream constructor methodology of the exemplary embodiment comprises imaging each of the frames of a feed, and then applying a transformation to each of these images. However, the transformation could be applied to imaging feeds having a different construction or format. The transformation may also be applied directly to the feeds.

The mask sprite may be provided as a set of instructions indicating a set of pixels whose values should be set to zero or set to a specified value (for example, indicating a particular colour). The instructions may comprise a matrix for transforming the pixel values of the image.

The user interface 200 is described as being split into four equal quarters when carrying four video streams in the multi-feed display mode. It should be understood that a different number of imaging feeds can be used, and the imaging feeds can be arranged in alternative configurations. The feeds may all be displayed as an equal size on user interface 200, or they may be assigned different sizes. This may be based, for example on the importance of the contents or the feed, their magnification and/or level of detail they contain. The imaging feeds do not necessarily originate from different devices, but rather each device may send more than one imaging feed or none.

Each computer device may take a wide range of forms, for example each may be provided as a desktop, laptop, tablet, mobile telephone, or alternative computer device.

The description refers to a user performing actions such as making selections on a user interface via computer mouse clicks. It should be understood that alternative methods of implementing actions such as selections on a user interface may be used. For example, an interactive touchscreen display, keyboard shortcuts and/or alternative controller may be used. In these implementations, the ‘clicks’ can be implemented via equivalent actions.

The computer devices of the present invention have been described each as comprising an authorization module. However, one or more ‘central’ authorization modules or devices may be provided, which define the authorizations of the different network computer devices. A central authorization module may be provided at the server or on one of the computer devices of the network. An ‘authorization module’ may be provided as any device, machine, entity and/or means for authorizing.

It will be understood that the present invention has been described above purely by way of example, and modifications of detail can be made within the scope of the invention.

Reference numerals appearing in the claims are by way of illustration only and shall have no limiting effect on the scope of the claims.

The term ‘comprising’ as used in this specification and claims preferably means ‘consisting at least in part of’. When interpreting statements in this specification and claims which include the term ‘comprising’, other features besides the features prefaced by this term in each statement can also be present. Related terms such as ‘comprise’ and ‘comprised’ are to be interpreted in a similar manner.

1. An apparatus for processing an imaging feed from an imaging device, comprising:
a device that is configured to transform the imaging feed by obscuring regions of the imaging feed;
a communication interface that is configured to communicate the imaging feed to a further device; and
an authorization module configured to authorize an entity to control the imaging feed before the imaging feed is communicated to the further device,
wherein, after the imaging feed is transformed, only the transformed imaging feed is transmitted beyond the communication interface, and
wherein the authorization module is configured to prohibit an entity from being able to view the imaging feed before the imaging feed is communicated to the further device,
wherein the authorization module is configured to authorize the same entity to define the transformation of the imaging feed.

2. The apparatus of claim 1, wherein the further device is external to the communication interface.

3. The apparatus of claim 1, wherein the further device is a server and wherein the server is configured to distribute the transformed imaging feed.

4. (canceled)

5. The apparatus of claim 1, wherein the imaging feed is not recorded prior to transformation by the transforming device, and wherein the imaging feed is transmitted from the imaging device to the transforming device without being recorded.

6. (canceled)

7. (canceled)

8. (canceled)

9. The apparatus of claim 1, wherein the authorization module is configured to authorize the entity to view or edit the imaging feed before the imaging feed is communicated to the further device.

10. The apparatus of claims 1, wherein the authorization module is configured to authorize the entity to define the transformation of the imaging feed before the imaging feed is communicated to the further device.

11. The apparatus of claim 1, wherein the authorization module is configured to prohibit an entity being able to control the imaging feed before the imaging feed is communicated to the further device.

12. (canceled)

13. The apparatus of claim 1, wherein the authorization module is configured to determine a level of authorization of the entity in dependence on a status of the entity.

14. The apparatus of claim 1, wherein the authorization module is configured to authorize the entity that controls the communication interface to view the imaging feed before the imaging feed is communicated to the further device.

15. (canceled)

16. The apparatus of claim 1, further comprising a processor for: extracting at least one region of the imaging feed and replacing the at least one region of the imaging feed with at least one region of an alternative image to generate the transformed imaging feed.

17. (canceled)

18. The apparatus of claim 16, wherein the at least one region of the alternative image spatially corresponds to the at least one region of the imaging feed.

19. The apparatus of claim 16, wherein the alternative image is an image of the imaging feed, to which the processor is configured to apply a filter.

20. The apparatus of claim 19, wherein the processor is configured to apply the filter to a larger portion of the image than the at least one region of the alternative image.

21. The apparatus of claim 19, wherein the filter is an obscuring filter.

22. The apparatus of claim 21, wherein the filter is selected in dependence on a performance metric of the apparatus, and wherein the performance metric relates to processing capability.

23. The apparatus of claim 1, wherein the imaging feed is live, or wherein transformation of the imaging feed is performed in real time to generate a live transformed imaging feed, and wherein the transformation is performed within 500 ms of receiving the imaging feed.

24. The apparatus of claim 1, further comprising a user interface configured to enable a user to define or edit the transformation applied to the imaging feed.

25. (canceled)

26. (canceled)

27. (canceled)

28. The apparatus of claim 1, further configured to output an alert upon detecting that the imaging device is capable of altering a configuration of the imaging feed.

29. (canceled)

30. (canceled)

31. A method of processing an imaging feed from an imaging device, comprising:
transforming the imaging feed by obscuring regions of the imaging feed;
communicating only a transformed imaging feed to a further device; and
authorizing an entity to control the imaging feed before the transformed imaging feed is communicated to the further device, comprising:
prohibiting an entity from being able to view the imaging feed before the imaging feed is communicated to the further device, and
authorizing the same entity to define a transformation of the imaging feed.

32. (canceled)

33. (canceled)

34. A non-transitory computer-readable medium comprising instructions which, when executed by a computer, cause the computer to perform operations comprising:
transforming an imaging feed by obscuring regions of the imaging feed;
communicating only a transformed imaging feed to a further device; and
authorizing an entity to control the imaging feed before the imaging feed is communicated to the further device, comprising:
prohibiting an entity from being able to view the imaging feed before the imaging feed is communicated to the further device, and
authorizing the same entity to define the transformation of the imaging feed.
